(19) United States
(12) Patent Application Publication
(10) Pub. No.: US 2025/0275904 A1
(43) Pub. Date: Sep. 4, 2025

(54) ANTI-SUN COSMETIC COMPOSITION CONTAINING MYCOSPORINE-LIKE AMINO ACIDS

(71) Applicants: Jean-Noël THOREL, Paris (FR); NAOS INSTITUTE OF LIFE SCIENCE, Paris (FR)

(72) Inventor: Jean-Noël THOREL, Paris (FR)

(73) Assignees: Jean-Noël THOREL, Paris (FR); NAOS INSTITUTE OF LIFE SCIENCE, Paris (FR)

(21) Appl. No.: 18/568,616

(22) PCT Filed: Jul. 7, 2022
(86) PCT No.: PCT/FR2022/051365

§ 371 Date: Dec. 8, 2023

(30) Foreign Application Priority Data
Jul. 8, 2021 (FR) 2107441

(51) Int. Cl. A61K 8/44; A61K 8/49; A61Q 17/04

(52) U.S. Cl. CPC A61K 8/44; A61K 8/49; A61Q 17/04; A61K 2800/592; A61K 2800/74

(57) ABSTRACT

The present invention relates to a composition and to the use thereof for protecting against ultraviolet solar radiation. In particular, the invention relates to a composition comprising:—at least one mycosporin-like amino acid, referred to as “MAA”;—at least two organic screening agents chosen from UVB organic screening agents and/or broad-spectrum organic screening agents which absorb both in the UVB range and the UVA range;—at least one UVA organic screening agent.

### FIELD OF THE INVENTION

The present invention relates to a composition, in particular a cosmetic or dermatological composition, advantageously solar, comprising mycosporin-like amino acids and solar screening agents, as well as its use to protect the skin against ultraviolet radiation.

### PRIOR STATE OF THE ART

Ultraviolet (UV) radiation consists of light with a wavelength between 100 nm and 400 nm, traditionally divided into 3 subgroups: UVA (400-315 nm), UVB (315-280 nm) and UV-C (280-100 nm). UV-C, of short wavelength, is the most high-energy and harmful UV. It is screening agented by the ozone layer of the atmosphere and does not reach the Earth's surface in considerable quantities. Most skin damage is caused by UVA and UVB.

UVB induces production of the skin's natural pigment called “melanin”, which is the origin of the phenomenon commonly called “tanning”. UVB also stimulates cells to produce thicker epidermis. The reactions described above constitute a defense mechanism of the body against UV radiation.

The high energy of UVB generates molecular disorders (DNA alteration and protein damage) which, in the long term, saturate and block the nuclear DNA repair system. This leads to permanent mutations in the genome of affected cells, which are the cause of skin cancer. In this case, it is a direct UVB toxicity. As for UVA, it is known that it penetrates the deeper layers of the skin, where it produces harmful effects, particularly within connective tissue and on blood vessels. In particular, it is the cause of the phenomenon called “helioderma”, i.e., the premature actinic aging of the skin.

In addition, although UVB is the main cause of skin cancer, UVA also contributes indirectly to this type of damage. Indeed, UVA and UVB are the cause of production of free radicals, in particular ROS (Reactive Oxygen Species). These are very unstable molecules with a very short half-life, in the order of a nanosecond to a millisecond. ROS are capable of damaging intracellular structures (DNA, membranes, intracellular proteins, etc.) as well as extracellular structures (components of the extracellular matrix such as collagen fibers, etc.). ROS also exert an indirect harmful action, by causing the oxidation of membrane lipids, which induces the formation of reactive carbonyl species that contribute to amplifying tissue and cellular damage.

Therefore, it is necessary to combat the harmful effects of UV radiation, particularly UVA and UVB, taking into account interindividual differences such as skin type (phototype) and sun exposure times. For this reason, anti-sun compositions have been developed to ensure photoprotection of areas subject to UV radiation. These compositions are in the form of lotion, oil, oil-in-water or water-in-oil type emulsion, foam, gel, stick or spray. They contain a cosmetically acceptable support and one or more chemical screening agents or mineral screens at various concentrations.

Mineral sunscreens, including zinc oxides and titanium dioxides, protect against UV rays by reflecting sunlight. Therefore, they act like mirrors located on the surface of the skin. Mineral screens, at least in micrometric formulations, are safe for human use and pose no health risks.

However, they have disadvantages:
mineral screens tend to dry out the skin and spread with difficulty, leaving white streaks on the skin that can be very unsightly;
the galenic assessment mineral screen-based compositions is often poor, particularly in silicone-free compositions.

Chemical screening agents are organic compounds that absorb light, creating a screening agenting layer on the skin that neutralizes UV rays. Thus, when the solar screening agent molecule receives UV radiation, it goes into an excited state by absorbing the energy of the radiation and then returns to its stable state. By this mechanism, light radiation is converted into heat and dissipated. Therefore, chemical screening agents act in the same way as melanin, the skin's natural photoprotective pigment. Most chemical or organic screening agents are lipophilic and are, therefore, suitable for use in highly water-resistant compositions. From a galenic point of view, organic screening agent-based compositions are generally more appreciated than mineral screen-based compositions, thereby making it possible to improve compliance by users.

In Europe, all organic screening agents or mineral screens, authorized by regulations, are included in a list (Annex VI of the European Cosmetic Products Directive). This is a list currently containing approximately thirty compounds according to their INCI designation which the cosmetics industry must draw from in order to formulate sun protection products. However, this list is not static and is constantly evolving based on the results of scientific research. Indeed, several studies have demonstrated that some of the organic screening agents on this list could cause risks to human health and the environment, particularly because they are suspected of acting as endocrine disruptors.

In particular, scientific results cast doubt on the safety of benzophenone-4 or even benzophenone-3 (Kinnberg et al. 2015; Zucchi et al. 2011), octyl methoxycinnamate (Szwarcfarb et al. 2008, Axelstad et al. 2011) 3-methylbenzylidene camphor and 4-methylbenzylidene camphor (Schlumpf et al. 2008; Petersen et al. 2007), octocrylene, isoamyl methoxycinnamate, or else octyl dimethyl PABA (Schlumpf et al. 2001; Rehfeld et al. 2016; Ozáez et al. 2016).

Alongside the possible effects on humans, the effect of these molecules on natural environments cannot be overlooked; indeed, the screening agents are continuously dispersed in bathing water or in wastewater. This water pollution, associated with UV screening agents, inevitably has repercussions throughout the food chain, with unpredictable consequences on the balance of an ecosystem already compromised by human activities; among other examples of ecotoxic effects, solar screening agents would have a negative impact on marine reefs and would be particularly responsible for the phenomenon of coral bleaching (Danovaro et al. 2008).

The gradual reduction in the number of organic solar screening agents, considered safe for human use and for the environment, increasingly complicates the formulation of cosmetic sunscreen products, particularly sunscreen products with a high protection factor, i.e., with an SPF (“Sun Protection Factor”) greater than 20 or even 30. The formulation of a product with high sun protection is particularly difficult in the case of products made for the Canadian and American market, where only part of the latest generation screening agents used in Europe for two decades is authorized.

To compensate for the reduced number of usable solar screening agents, sun protection agents are often formulated in cosmetic products. These sun protection agents are sometimes molecules close to authorized solar screening agents, or plastic derivatives, which can, therefore, suffer from the same disadvantages as organic solar screening agents; in particular, their ecotoxicity could be significant.

An interesting family of protective agents is represented by Mycosporin-like Amino Acids (or MAAs). MAAs are small heterocyclic molecules (<400 Da) comprising cyclohexenone or cyclohexenimine chromophores.

MAAs are found naturally as secondary metabolites in marine organisms, such as corals, algae and cyanobacteria, and are, therefore, expected to be eco-compatible and respectful of aquatic ecosystems.

In particular, document EP2855441 discloses a family of compounds, of the MAA type, having the following general IE formula:

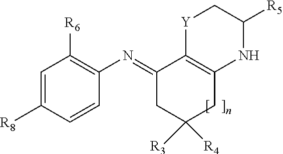

That document specifies that these compounds, from the IE molecule family, absorb ultraviolet radiation and may be used as an anti-UV agent. The compounds of formulas IA1 and IE4 having a sun protection factor of 2.4 and 4.5, respectively, are given as examples.

Thus, although the MAAs of the IE molecule family absorb UV radiation, their sun protection factor is insufficient for use in anti-sun cosmetic compositions.

Although considered good candidates for the formulation of eco-compatible solar products, a solution must be found to improve the anti-UV potential of MAAs, in order to be able to use them in solar products.

Thus, the present invention aims to overcome the disadvantages of the state of the art, by proposing an ecobiological composition comprising MMAs and whose SPF is compatible with topical solar application.

### DISCLOSURE OF THE INVENTION

The applicant observed, quite surprisingly, that combining MMAs with at least two organic UVBs and/or broad-spectrum screening agents in the presence of at least one organic UVA screening agent made it possible to obtain an SPF with a value greater than that of the sum of the respective SPFs of the MAAs and organic screening agents.

Therefore, this combination lends itself to their implementation in compositions with high solar protection with relatively low levels of organic screening agents.

Thus, according to a first aspect, the invention relates to a composition, in particular a cosmetic or dermatological composition, comprising:
at least one amino acid analog of mycosporin called “MAA”;
at least two organic UVB screening agents and/or at least two broad-spectrum organic screening agents;
at least one organic UVA screening agent.

According to another aspect, the invention relates to a composition, in particular a cosmetic or dermatological composition, comprising:
at least one amino acid analog of mycosporin called “MAA” chosen from the group consisting of molecules A of formula IA or one of its acceptable salts, and/or the group consisting of molecules

B of formula IB or one of its acceptable salts, said formula IA being:

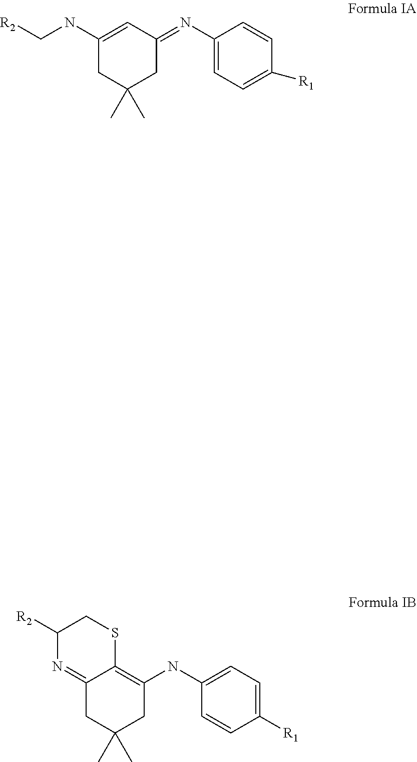

wherein:
R1 is hydrogen; alkyl; alkenyl; alkynyl; aryl; heterocycle; cycloalkyl; alkoxy; alkanoyl; hydroxyl; a sulfo group; a halogen group; a phosphono group; an ester group; a carboxylic acid group; a phenyl group; an amino group; an alkylated fatty acid chain or a polyether;
R2 is alkyl; alkenyl; alkynyl; aryl; heterocycle; cycloalkyl; alkoxy; alkanoyl; a sulfo group; a phosphono group; an ester group; a carboxylic acid group; hydroxyl; or a phenyl group; said composition further comprising:
at least two organic screening agents chosen from organic UVB screening agents and/or broad-spectrum organic screening agents absorbing both UVBs and UVAs;
at least one organic UVA screening agent.

According to a particular embodiment, the composition according to the invention is ecobiological. The term “ecobiological composition” is understood to mean respectful of the person, their interactions with the world, and the planet.

The MAAs according to the invention are known to a person skilled in the art and disclosed, for example, in patent application WO2013/181741 which details the process for obtaining and purifying them.

According to a particular embodiment, R1 is an alkoxy group of the [CH₃]—[CH₂]ₙ—O— type, wherein “n” is between 0 and 10. According to a preferred embodiment, “n”=0 or “n”=8.

According to a particular embodiment of the invention, R2 is a carboxylic acid group of the type [COOH]—[CH₂]ₙ—, wherein “n” is between 0 and 5, or preferably “n”=1 or “n”=0.

According to an alternative embodiment of the invention, R2 is an ester group of the type [CH₃]—[CH₂]ₙ—[COOH]—, wherein “n” is between 0 and 5, or preferably “n”=1 or “n”=2.

According to another particular embodiment, R1 is an alkoxy group of the [CH₃]—[CH₂]ₙ—O— type, where “n”=0; R2 is an ester group of the type [CH₃]—[CH₂]—[COOH]—, where “n”=1 or “n”=2 or R2 is a carboxylic acid group of the type [COOH]—[CH₂]ₙ—, wherein “n” is between 0 and 5, or preferably “n”=1 or “n”=0.

Thus, and according to a particular embodiment, the MAA according to formula IA corresponds to the molecule of formula IIA1 below:

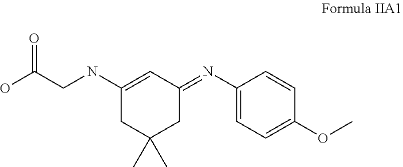 [SMILES: COc1ccc(/N=C2/C=C(NCC(=O)O)CC(C)(C)C2)cc1]

This molecule of formula IIA1: [N-[(3E)-3-[(4-methoxyphenyl)imino]-5,5-dimethyl-1-cyclohexen-1-yl]-glycine, corresponding to CAS number 1509902-01-5] is marketed by ELKIMIA Inc. under the INCI designation: methoxyphenylimino dimethylcyclohexene glycine.

In another embodiment, the MAA according to formula IA corresponds to the molecule of formula IIA2 below:

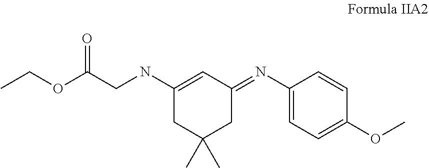 [SMILES: CCOC(=O)CNC1=C/C(=N/c2ccc(OC)cc2)CC(C)(C)C1]

This molecule of formula IIA2: [N-[3-[(4-methoxyphenyl)amino]-5,5-dimethyl-2-cyclohexen-1-ylidene]-, ethyl ester, [N (E)]-glycine, corresponding to CAS number 2640340-86-7] is available from SENSIENT Inc. under the trade name SENSISORB™ BIOMIM (INCI: methoxyphenylimino dimethylcyclohexenyl ethyl glycinate).

According to a preferred embodiment, the MAA according to formula IB corresponds to the molecule of formula IIB1 below:

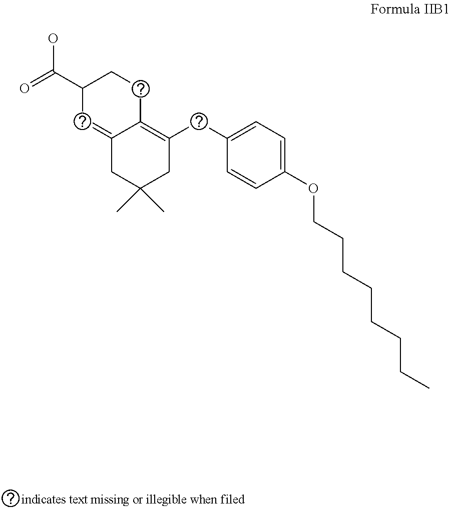 [SMILES: CCCCCCCCOc1ccc(CC2=C3CCC(C(=O)O)C=C3CC(C)(C)C2)cc1]

This molecule of formula IIB1 [3,5,6,7-tetrahydro-6,6-dimethyl-8-[4-(octyloxy)phenyl]amino]-2H-1,4-Benzothiazine-3-carboxylic acid, corresponding to CAS number 1629023-01-3] is marketed by ELKIMIA Inc. under the INCI designation: caprylyloxyphenylamino dimethyltetrahydro benzothiazine carboxylic acid.

According to an alternative embodiment, the MAA according to formula IB corresponds to the molecule of formula IIB2 below:

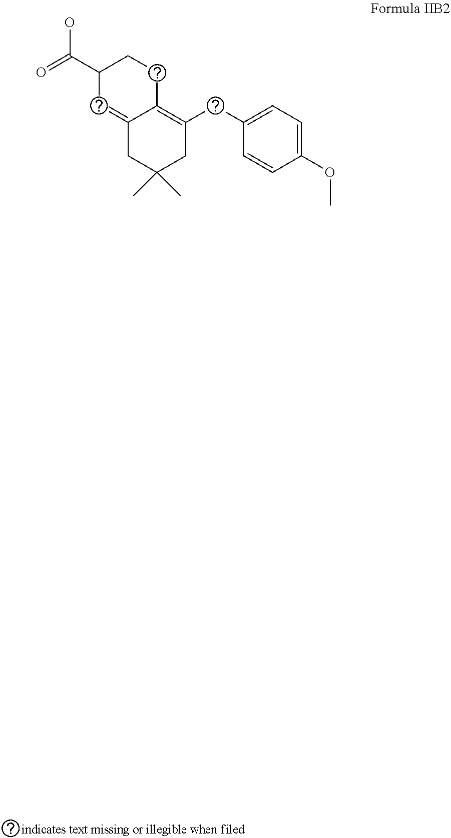 [SMILES: COc1ccc(CC2=C3CCC(C(=O)O)C=C3CC(C)(C)C2)cc1]

This molecule of formula IIB2 [3,5,6,7-tetrahydro-8-[(4-methoxyphenyl)amino]-6,6-dimethyl-2H-1,4-Benzothiazine-3-carboxylic acid corresponding to CAS number 1629023-04-6] is marketed by ELKIMIA Inc. under the INCI designation: methoxyphenylimino dimethylhexahydro benzothiazine carboxylic acid.

According to an alternative embodiment, the MAA according to formula IB corresponds to the molecule of formula IIB3 below.

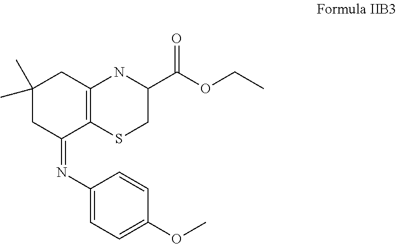 [SMILES: CCOC(=O)C1CSC2=C(CC(C)(C)C/C2=N/c2ccc(OC)cc2)N1]

This molecule of formula ethylester-(3R)-2H-1,4-Benzothiazine-3-carboxylic acid, 3,4,5,6,7,8-hexahydro-8-[(4-methoxyphenyl)imino]-6,6-dimethyl corresponds to CAS number 2699128-33-9.

In a particular embodiment of the invention, the compositions according to the invention contain at least two MAAs as defined above, in particular chosen from the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid and methoxyphenylimino dimethylcyclohexenyl ethyl glycinate as well as, optionally, the compound corresponding to CAS number 2699128-33-9.

According to a particular embodiment, the compositions according to the invention contain at least three MAAs.

Within the meaning of the invention, the term “acceptable salts” is understood to mean any pharmacologically or cosmetically acceptable salt of MAA, such as, for example,, sulfate, citrate, acetate, oxalate, chloride, bromide, iodide, nitrate, bisulfate, phosphate, isonicotinate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, pamoate and 3-hydroxynaphthoate salts.

According to a particular embodiment, the MAA(s) represent(s) between 0.1% and 20% by mass relative to the total mass of the composition, preferably between 0.5% and 10%, even more advantageously between 1 and 2%.

The term “organic solar screening agent” designates organic UVB screening agents, organic UVA screening agents and broad-spectrum organic screening agents.

Within the meaning of the invention, the term “organic UVB screening agent” is understood to mean any organic solar screening agent, whether hydrophilic or lipophilic, which, mainly or exclusively, absorbs UVBs.

According to a particular embodiment, the organic UVB screening agent is chosen from salicylates.

More particularly, the organic UVB screening agent is chosen from the compounds corresponding to the following INCI designations: ethylhexyl salicylate and homosalate or else phenylbenzimidazole sulfonic acid or ethylhexyl triazone.

A source of the latter is the raw material UVINUL™ T150 marketed by BASF.

The organic UVB screening agent corresponding to the INCI designation: phenylbenzimidazole sulfonic acid, for its part, is available from AAKO BV under the name AakoSun PBSA. Salicylates corresponding to the INCI designations: octyl salicylate and homosalate are available from SYMRISE under the names Neo Heliopan™ OS and Neo Heliopan™ HMS, respectively.

According to a particular embodiment of the invention, said at least one organic UVB screening agent represents between 1% and 20% by mass relative to the total mass of the composition, preferably between 2% and 15%.

Within the meaning of the invention, the term “organic UVA screening agent” is understood to mean any organic solar screening agent, whether hydrophilic or lipophilic, which, mainly or exclusively, absorbs UVAs.

According to a preferred embodiment, the composition according to the invention comprises at least one UVA solar screening agent chosen from the group comprising the compounds corresponding to the following INCI designations: butyl methoxydibenzoylmethane, diethylamino hydroxybenzoyl hexyl benzoate, bis-(diethylaminohydroxybenzoyl benzoyl) piperazine, disodium phenyl dibenzimidazole tetrasulfonate, especially butyl methoxydibenzoylmethane and diethylamino hydroxybenzoyl hexyl benzoate.

For example, the UVA screening agents according to the invention correspond to the raw material Parsol 1789® (INCI: butyl methoxydibenzoylmethane) marketed by DSM; the raw material UVINUL™ A+ (INCI: diethylamino hydroxybenzoyl hexyl benzoate) marketed by BASF; the raw material C1332™ [INCI: bis-(diethylaminohydroxybenzoyl benzoyl) piperazine; CAS number 919803-06-8] marketed by BASF; or the raw material Neo Heliopan™ AP corresponding to the INCI designation: disodium phenyl dibenzimidazole tetrasulfonate, and marketed by SYMRISE.

According to a particular embodiment, said at least one organic UVA screening agent represents between 1% and 10% by mass relative to the total mass of the composition, preferably between 2% and 7%.

Within the meaning of the invention, the term “broad-spectrum organic screening agent” is understood to mean any organic solar screening agent, whether hydrophilic or lipophilic, which absorbs both UVBs and UVAs. According to a preferred embodiment, the composition according to the invention comprises at least one broad-spectrum solar screening agent chosen from the group comprising the compounds corresponding to the following INCI designations: bis-ethylhexyloxyphenol methoxyphenyl triazine, diethylhexyl butamido triazone, tris-biphenyl triazine, phenylene bis-diphenyltriazine, methylene bis-benzotriazolyl tetramethylbutylphenol and drometrizole trisiloxane, in particular bis-ethylhexyloxyphenol methoxyphenyl triazine, diethylhexyl butamido triazone, tris-biphenyl triazine, methylene bis-benzotriazolyl tetramethylbutylphenol.

These solar screening agents are available from the following suppliers:
PARSOL™ Shield marketed by SYMRISE and corresponding to the INCI designation: bis ethylhexyloxyphenol methoxyphenyl triazine;
UVASORB™ HEB marketed by SIGMA 3V and corresponding to the INCI designation: diethylhexyl butamido triazone;
TINOSORB™ A2B marketed by BASF and corresponding to the INCI designation: tris-biphenyl triazine;
TRIASORB™ marketed by PLANTES & INDUSTRIE and corresponding to the INCI designation: phenylene bis-diphenyltriazine (CAS number 55514-22-2);
MEXORYL™ XL, corresponding to the INCI designation: drometrizole trisiloxane;
TINOSORB™ M marketed by BASF and corresponding to the INCI designation: methylene bis-benzotriazolyl tetramethylbutylphenol.

According to a particular embodiment, said at least one broad-spectrum organic screening agent represents between 1% and 15% by mass relative to the total mass of the composition, preferably between 2% and 10%.

According to a particular embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9,
at least two organic UVB screening agents;
at least one organic UVA screening agent.

According to an alternative embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two broad-spectrum organic screening agents;
at least one organic UVA screening agent.

According to an alternative embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least one organic UVB screening agent;
at least one broad-spectrum organic screening agent;
at least one organic UVA screening agent.

According to yet another embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least one organic UVB screening agent;
at least two broad-spectrum organic screening agents;
at least one organic UVA screening agent.

According to yet another embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two organic UVB screening agents;
at least one broad-spectrum organic screening agent;
at least one organic UVA screening agent.

According to another aspect, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least 2 UVB screening agents corresponding to the following INCI designations: octyl salicylate and homosalate;
at least the organic UVA screening agent corresponding to the following INCI designation: butyl methoxydibenzoylmethane.

According to another embodiment, the composition according to the invention comprises as MAA and organic solar screening agents:
one MAA chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9, advantageously methoxyphenylimino dimethylcyclohexene glycine;
2 UVB screening agents corresponding to the following INCI designations: octyl salicylate and homosalate;
an organic UVA screening agent corresponding to the following INCI designation: butyl methoxydibenzoylmethane.

According to another embodiment, the composition according to the invention comprises as MAAs and organic solar screening agents:
two MAAs chosen from the group consisting of molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9, advantageously methoxyphenylimino dimethylcyclohexene glycine and methoxyphenylimino dimethylhexahydro benzothiazine carboxylic acid and methoxyphenylimino dimethylcyclohexenyl ethyl glycinate;
2 UVB screening agents corresponding to the following INCI designations: octyl salicylate and homosalate;
an organic UVA screening agent corresponding to the following INCI designation: butyl methoxydibenzoylmethane.

According to a particular embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least 2 UVB screening agents, corresponding to the following INCI designations: octyl salicylate and homosalate;
at least the organic UVA screening agent corresponding to the following INCI designation: diethylamino hydroxybenzoyl hexyl benzoate.

According to a different embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two broad-spectrum screening agents corresponding to the following INCI designations: diethylhexyl butamido triazone and bis-ethylhexyloxyphenol methoxyphenyl triazine;
at least one UVA solar screening agent chosen from the group comprising the compounds corresponding to the following INCI designations: butyl methoxydibenzoylmethane, diethylamino hydroxybenzoyl hexyl benzoate, bis-(diethylaminohydroxybenzoyl benzoyl) piperazine, disodium phenyl dibenzimidazole tetrasulfonate, in particular butyl methoxydibenzoylmethane and diethylamino hydroxybenzoyl hexyl benzoate, advantageously butyl methoxydibenzoylmethane and diethylamino hydroxybenzoyl hexyl benzoate.

According to a different embodiment, the composition according to the invention comprises:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two broad-spectrum screening agents corresponding to the following INCI designations: diethylhexyl butamido triazone and bis-ethylhexyloxyphenol methoxyphenyl triazine;
at least one UVA solar screening agent corresponding to the following INCI designation: diethylamino hydroxybenzoyl hexyl benzoate.

According to another embodiment, the composition according to the invention comprises as MAA and organic solar screening agents:
one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the corresponding compound with CAS number 2699128-33-9, advantageously methoxyphenylimino dimethylcyclohexene glycine;
two broad-spectrum screening agents corresponding to the following INCI designations: diethylhexyl butamido triazone and bis-ethylhexyloxyphenol methoxyphenyl triazine;
a UVA solar screening agent corresponding to the following INCI designation: diethylamino hydroxybenzoyl hexyl benzoate.

According to an alternative embodiment, the composition according to the invention comprises:
at least one MAA as defined above, in particular chosen from the group consisting of the corresponding molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least broad-spectrum screening agents corresponding to the following INCI designations: diethylhexyl butamido triazone and bis-ethylhexyloxyphenol methoxyphenyl triazine;
at least the organic UVB screening agent corresponding to the following INCI designation: ethylhexyl triazone;
at least the organic UVA screening agent corresponding to the following INCI designation: butyl methoxydibenzoylmethane.

According to a particular embodiment, the composition according to the invention comprises:
at least one MAA as defined above, in particular chosen from the group consisting of the corresponding molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate as well as, optionally, the compound corresponding to CAS number 2699128-33-9;
at least broad-spectrum organic screening agents corresponding to the following INCI designations: diethylhexyl butamido triazone and bis-ethylhexyloxyphenol methoxyphenyl triazine;
at least the organic UVB screening agent corresponding to the following INCI designation: ethylhexyl triazone;
at least the organic UVA screening agent corresponding to the following INCI designation: diethylamino hydroxybenzoyl hexyl benzoate.

According to a particular embodiment, the composition according to the invention is free from at least one, alternatively several solar screening agents corresponding to the following INCI designations: 4-methylbenzylidene camphor, 3-methylbenzylidene camphor, benzophenone-2, benzophenone-3, benzophenone-4, ethylhexyl methoxycinnamate, isoamyl methoxycinnamate, octocrylene, octyl dimethyl PABA.

Preferably, the composition also includes at least one solar screening agent solubilizer. For the purposes of the invention, the term “solubilizer” is understood to mean a compound which makes it possible to solubilize, disperse and/or dissolve at least one solar screening agent derived from triazine in an effective and sustainable manner, i.e., by stabilizing it in its solubilized form while preventing or reducing its recrystallization or precipitation in formulation throughout the duration of use of the product.

Thus, the compositions according to the invention further comprise at least one solubilizer chosen from the group comprising the compounds corresponding to the following INCI designations: caprylyl caprylate/caprate, dibutyl adipate, dicaprylyl carbonate, diisopropyl sebacate, dicaprylyl ether, coco-caprylate, C12-15 alkyl benzoate, propylheptyl caprylate, butylene glycol dicaprylate/dicaprate, dipropylene glycol dibenzoate, neopentyl glycol diheptanoate, triheptanoin, C12-13 alkyl lactate, ethylhexyl benzoate, C12-C15 alkyl lactate, C12-13 alkyl tartrate, tridecyl salicylate, lauryl lactate, diethyl adipate, diisobutyl adipate, diisopropyl adipate, diethylhexyl adipate, diethylhexyl succinate, propanediol dicaprylate, propylene glycol dicaprylate/dicaprate, isopropyl lauroyl sarcosinate, propylene glycol dibenzoate, hydroxyl dimethoxybenzyl malonate, phenoxyethyl caprylate, isodecyl salicylate, dimethyl capramide and phenethyl benzoate, in particular at least two solubilizers chosen from the following group: caprylyl caprylate/caprate, dibutyl adipate, dicaprylyl carbonate, diisopropyl sebacate, dicaprylyl ether, coco-caprylate, C12-15 alkyl benzoate, propylheptyl caprylate, butylene glycol dicaprylate/dicaprate.

Solubilizers capable of being used in a composition according to the invention are available on the market from several suppliers. By way of example, the following raw materials may be used in the composition according to the invention:
Several raw materials from the CETIOL™ range marketed by BASF, in particular CETIOL™ RLF, CETIOL™πB, CETIOL™ CC, CETIOL™ O, CETIOL™ C5, CETIOL™ AB, CETIOL™ SENSOFT corresponding respectively to the following INCI designations: caprylyl caprylate/caprate, dibutyl adipate, dicaprylyl carbonate, dicaprylyl ether, coco-caprylate, C12-15 alkyl benzoate, propylheptyl caprylate;
DUB™ DIS, DEA, DIBA, ZENOAT marketed by STEARINE DUBOIS corresponding respectively to the following INCI designations: diisopropyl sebacate, diethyl adipate, diisobutyl adipate and propanediol dicaprylate;
MIGLYOL™ 8810 and MIGLYOL™ T-C7 marketed by IOI Oleo GmbH corresponding respectively to the following INCI designations: butylene glycol dicaprylate/dicaprate and triheptanoin;
Lexsolv™ A marketed by INOLEX corresponding to the following INCI designations: dipropylene glycol dibenzoate and neopentyl glycol diheptanoate;
COSMACOL™ ELI, ETI, ESI and LL marketed by SASOL and corresponding respectively to the following INCI designations: C12-13 alkyl lactate, C12-13 alkyl tartrate, tridecyl salicylate and lauryl lactate;
Ceraphyl™ 41 ester marketed by ASHLAND and corresponding to the following INCI designation: C12-C15 alkyl lactate;
Finsolv™ EB and PG 22 marketed by INNOSPEC and corresponding respectively to the following INCI designations: ethylhexyl benzoate and dipropylene glycol dibenzoate;
CRODAMOL™ DA, DOA, OSU and PC marketed by CRODA and corresponding respectively to the following INCI designations: diisopropyl adipate, diethylhexyl adipate, diethylhexyl succinate and propylene glycol dicaprylate/dicaprate;
ELDEW™ SL-205 marketed by AJINOMOTO and corresponding to the following INCI designation: isopropyl lauroyl sarcosinate;
Lexfeel™ Shine marketed by INOLEX and corresponding to the INCI designation: propylene glycol dibenzoate;
TEGOSOFT™ XC marketed by EVONIK and corresponding to the following INCI designation: phenoxyethyl caprylate;
RONACARE™ AP marketed by MERCK KGaA and corresponding to the following INCI designation: hydroxyl dimethoxybenzyl malonate;
DERMOL™ IDSA marketed by Alzo INTL and corresponding to the following INCI designation: isodecyl salicylate;
Spectrasolv™ DMDA marketed by Hallstar and corresponding to the following INCI designation: dimethyl capramide;
X-TEND™ 226 marketed by Ashland and corresponding to the following INCI designation: phenethyl benzoate.

According to a particular embodiment, the composition according to the invention comprises at least three solubilizers chosen from the group comprising the compounds corresponding to the following INCI designations: caprylyl caprylate/caprate, dibutyl adipate, dicaprylyl carbonate, diisopropyl sebacate, dicaprylyl ether, coco-caprylate, C12-15 alkyl benzoate, propylheptyl caprylate, butylene glycol dicaprylate/dicaprate.

According to a particular embodiment, the composition according to the invention comprises at least the solubilizers corresponding to the following INCI designations: dibutyl adipate, dicaprylyl carbonate and diisopropyl sebacate.

According to another embodiment, the composition according to the invention comprises at least the solubilizers corresponding to the following INCI designations: dibutyl adipate, dicaprylyl carbonate, diisopropyl sebacate and propylheptyl caprylate.

According to a particular embodiment, the solubilizers represent between 5% and 80% by mass relative to the total mass of the composition, advantageously between 10% and 70%, preferably between 15% and 60%.

According to a particular embodiment, the composition according to the invention comprises mineral screens (or inorganic mineral screening agents), which correspond to metal oxides and/or other compounds that are poorly soluble or insoluble in water, in particular oxides titanium (TiO₂), zinc (ZnO), iron (Fe₂O₃), zirconium (ZrO₂), silicon (SiO₂), manganese (e.g., MnO), aluminum (Al₂O₃), or cerium (Ce₂O₃), or bismuth trioxide (Bi₂O₃).

Inorganic mineral screening agents may also be surface-treated or encapsulated in order to give them a hydrophilic, amphiphilic or hydrophobic character. This surface treatment may consist of providing the mineral screening agents with a thin hydrophilic and/or hydrophobic inorganic and/or organic film.

According to a particular embodiment, the composition according to the invention comprises at least one mineral screen chosen from the group comprising the compounds corresponding to the following INCI designations: zinc oxide, titanium dioxide and mixtures thereof.

For example, zinc oxide corresponds to the Z-COTE® LSA raw material and titanium dioxide to the T-Lite® raw material, marketed by BASF.

The lists of UV screening agents cited which may be implemented within the meaning of the present invention are, obviously, given by way of example but not limited thereto.

According to a particular embodiment, the composition according to the invention has a sun protection factor called “SPF” equal to or greater than 20, advantageously equal to or greater than 30, preferably equal to or greater than 40, or even equal to or greater than 50.

According to a preferred embodiment, the composition according to the invention comprises a UVA/UVB protection ratio equal to or greater than 1/3.

In particular, the composition according to the invention has a critical wavelength (λc) greater than 370 nm. This value, which is determined by in vitro methods known to a person skilled in the art, corresponds to the wavelength for which the integral of the absorption spectrum curve starting at 290 nm reaches 90% of the integral between 290 and 400 nm.

The composition according to the invention may further comprise an SPF “booster”, i.e., an amplifying agent of the sun protection factor, and/or a photo-stabilizer, i.e., an ingredient which makes it possible to increase the SPF or to photostabilize the screening agents, such an ingredient not itself being considered as a solar screening agent. These boosters may include, for example:
butyloctyl salicylate (INCI), photostabilizer advantageously representing between 0.01% and 10% by weight relative to the total weight of the composition, even more advantageously between 0.1% and 2%. This raw material is, for example, marketed by HALLSTAR under the name Hallbrite™ BHB;
benzotriazolyl dodecyl p-cresol (INCI), photostabilizer advantageously representing between 0.01% and 10% by weight relative to the total weight of the composition, even more advantageously between 0.1% and 2%. This raw material is, for example, marketed by BASF under the name TINOGARD™ TL;
pongamol (INCI), a plant molecule absorbing UVAs, advantageously representing between 0.5 and 2% by weight relative to the total weight of the composition, even more advantageously of the order of 1%. For example, the raw material Pongamia Extract marketed by GIVAUDAN may be used in the context of the present invention;
ethylhexyl methoxycrylene (INCI), photostabilizer, solubilizer and SPF “booster” advantageously representing between 1% and 5% by weight relative to the total weight of the composition. The SolaStay™ S1 raw material marketed by HALLSTAR may be used in the context of the present invention;
a styrene acrylate copolymer (INCI: styrene/acrylate copolymer), preferably representing between 1% and 10% by weight relative to the total weight of the composition according to the invention. The SunSpheres™ H53 and SunSpheres™ PGL Polymer raw materials, marketed by DOW CHEMICALS, may be used in the context of the present invention;
diethylhexyl syringylidene malonate (INCI), advantageously representing between 1% and 10% by weight relative to the total weight of the composition. The raw material OXYNET™ ST, marketed by MERCK, may be used in the context of the present invention;
a water-dispersible polyester, corresponding to the INCI designations: polyester-5 (and) Sodium silicoaluminate, advantageously representing between 1% and 10% by weight relative to the total weight of the composition, in particular EASTMANN AQTM38S Polymer marketed by SAFIC-ALCAN;
an acrylate copolymer having a glass transition temperature of −5° C. to −15° C. as measured by differential scanning calorimetry, said copolymer advantageously representing between 1% and 10% by weight relative to the total weight of the composition. For example, a polymer corresponding to the INCI designation: Acrylate copolymer, such as the raw material EPITEX 66, marketed by DOW CHEMICALS, may be used in the context of the present invention.

In a preferred embodiment, the composition according to the invention further comprises other components which may contribute to internal protection by an action which may consist of a DNA protection, a reduction in immunosuppression induced by UV radiation, an anti-radical action or a combined effect of these actions.

The protective action of a composition according to the invention against oxidative stress or against the effect of free radicals may be further improved if it further comprises one or more antioxidants, easily selected by a person skilled in the art, for example, in the following list: totarol, magnolol, honokiol, amino acids and their derivatives, peptides (D and/or L-carnosine) and their derivatives (for example anserine, 1 hypotaurine, taurine), carotenoids, carotenes (α-carotene, β-carotene, lycopene) and their derivatives, chlorogenic acid and its derivatives, lipoic acid and its derivatives (dihydrolipoic acid), aurothioglucose, propylthiouracil and other thiols (thioredoxin, glutathione, cysteine, cystine, cystamine and their glycosyl, N-acetyl, methyl, ethyl, propyl, amyl, butyl and lauryl, palmitoyl, oleyl, γ-linoleic, cholesteryl and glyceryl esters) as well as salts thereof, dilauryl thiodipropionate, distearyl thiodipropionate, thiodipropionic acid and its derivatives, sulfoximine compounds (buthionine sulfoximine, homocysteine sulfoximine, buthionine sulfones, penta-, hexa- and heptathionine sulfoximine), chelating agents (such as α-hydroxy fatty acids, palmitic acid, phytic acid, lactoferrin), α-hydroxy acids (such as citric, lactic, or malic acid), acid humic, bile acid, bile extracts, bilirubin, biliverdin, ethylenediamine pentasodium tetramethylene phosphonate and its derivatives, unsaturated fatty acids and their derivatives, vitamin A and its derivatives (vitamin A palmitate), coniferyl benzoate from benzoin resin, rutinic acid and its derivatives, α-glycosyl rutin, ferulic acid and its derivatives, furfurylideneglucitol, carnosine, butylated hydroxytoluene, butylated hydroxyanisole, nordihydroguaiaretic acid, trihydroxybutyrophenone, quercetin, uric acid and its derivatives, mannose and its derivatives, zinc and its derivatives (ZnO, ZnSO4), selenium and its derivatives (selenomethionine), stilbenes and their derivatives (1 stilbene oxide, trans-stilbene oxide).

In a particular embodiment, the composition according to the invention further comprises glycyrrhetinic acid, a derivative or salt of this acid, used as a soothing (anti-inflammatory agent) and representing between 0.01% and 2% by weight relative to the total weight of the composition, preferably between 0.1% and 1%.

According to another preferred characteristic of the invention, the cosmetic and/or dermatological composition comprises at least one, or even all, of the following constituents exerting a biological activity in vivo on the cells of the skin, lips, hair and/or mucous membranes subjected to UVA and/or UVB radiation, respectively:
an anti-radical agent preserving cellular structures, such as, for example, vitamin E and/or its lipid-soluble or water-soluble derivatives, in particular tocotrienol and/or tocopherol, advantageously representing between 0.001 and 10% by weight relative to the total weight of the composition, even more advantageously between 0.02 and 2%, preferably of the order of 0.04%;
an agent limiting immunosuppression, such as, for example, vitamin PP, advantageously representing between 0.001 and 1% by weight relative to the total weight of the composition, even more advantageously from 0.01% to 0.3%;
a protective agent for the p53 protein, such as, for example, epigallocatechin gallate (EGCG), advantageously representing between 0.001 and 0.1% by weight relative to the total weight of the composition, even more advantageously from 0.005% to 0.05%.

The composition according to the invention may further comprise, in addition, peptide extracts of soya and/or wheat, such as those described in document EP 2 059 230.

In practice, the peptide extracts, which come from soybean and wheat seeds, are the result of enzymatic hydrolysis of said seeds using peptidases which make it possible to recover peptides with an average size of 700 Daltons. Preferably, the soy peptide extract is the extract identified under the CAS number 68607-88-5, just as the wheat peptide extract is the extract identified under the CAS number 70084-87-6. Wheat and soy extracts may correspond respectively to the following INCI designations: hydrolyzed wheat protein and hydrolyzed soy protein.

In a particular embodiment, the peptide extracts of soya and/or wheat are used together, for example in a weight ratio respectively between 80/20 and 20/80, advantageously between 70/30 and 30/70, of preferably equal to 60/40.

In a particular embodiment, the peptide extracts of soy and/or wheat are free of synthetic GHK (glycyl-histidyl-lysine; INCI: Tripeptide-1) tripeptides. In practice, the peptide extracts of soya and/or wheat represent from 0.01 to 20% by weight relative to the total weight of the composition, advantageously from 0.1% to 10%, even more advantageously from 0.2% to 0.7%.

In an alternative embodiment, the composition according to the invention comprises, in accordance with the teachings of document FR 2 865 398, the association of at least one amino acid chosen from the group consisting of ectoine, creatine, ergothioneine and/or carnosine, or their physiologically acceptable salts, and mannitol or a mannitol derivative.

Preferably, the composition according to the invention comprises, in a physiologically acceptable medium, the amino acid or one of its salts, alone or as a mixture in proportions of between 0.001% and 10% by weight relative to the total weight of the composition, and preferably between 0.01% and 5%.

The composition according to the present invention preferably comprises, in a physiologically acceptable medium, mannitol or one of its derivatives, in proportions of between 0.01% and 30% by weight relative to the total weight of the composition, advantageously between 0.1% and 10%.

In a preferred embodiment, the composition according to the invention comprises ectoine and mannitol.

According to a particular embodiment, the composition according to the invention comprises one or more other tanning or self-tanning agents. It may be a self-tanner which reacts with the amino acids of the skin according to a Maillard reaction or through a Michael addition, or a promoter of melanogenesis or a propigmenting compound which promotes the natural tanning process of the skin.

Such a tanning or self-tanning agent is preferably present in the composition in an amount ranging from 0.01% to 20% by weight relative to the total weight of the composition, advantageously from 0.5% to 15%, even more advantageously from 1% at 8%.

Self-tanning substances may be 1,3-dihydroxyacetone (DHA), glycerolaldehyde, hydroxymethylglyoxal, γ-dialdehyde, erythrulose, 6-aldo-D-fructose, ninhydrin, 5-hydroxy-1,4-naphthoquinone (juglone), 2-hydroxy-1,4-naphthoquinone (lawsone), or a combination thereof.

The propigmenting substances may be melanocyte-stimulating hormone (α-MSH), peptide analogs of α-MSH, endothelin-1 receptor agonists, u-opioid receptor agonists, cAMP stimulators, or tyrosinase stimulators.

In a particular embodiment, the composition according to the invention further comprises, as a self-tanner, a combination of dihydroxy methylchromonyl palmitate and/or dimethylmethoxy chromanol, as well as a lipophilic form of tyrosine.

This combination of active ingredients effectively stimulates tanning. Dihydroxy methylchromonyl palmitate (CAS number: 1387636-35-2) corresponds, for example, to the cosmetic ingredient marketed by MERCK under the name RonaCare™ Bronzyl. Dimethylmethoxy chromanol (CAS number: 83923-51-7) corresponds, for example, to the cosmetic ingredient marketed by LIPOTEC SA under the name lipochromone-6.

According to a particular embodiment, dihydroxy methylchromonyl palmitate or dimethylmethoxy chromanol is included in the composition according to the invention in an amount of 0.01% to 10% by weight relative to the total weight of the composition, advantageously 0.05% to 10%, even more advantageously from 0.1% to 5%, more particularly from 0.1% to 0.5%.

Within the meaning of the invention, the lipophilic form of tyrosine is an tyrosine-based ingredient that has a more pronounced lipophilic character than tyrosine. The lipophilic form of tyrosine may, in particular, correspond to oleoyl tyrosine (CAS number: 147732-57-8), which is found, for example, in the liquid cosmetic ingredient TYR-OL, marketed by SEDERMA, and which comprises approximately 50% by weight of oleoyl tyrosine in butylene glycol (approximately 30%+approximately 20% oleic acid), or in the liquid cosmetic ingredient TYR-EXCEL, marketed by SEDERMA, which comprises approximately 50% by weight of oleoyl tyrosine, approximately 20% by weight of oleic acid (CAS No.: 112-80-1) and approximately 30% by weight of Luffa cylindrica oil (sponge pumpkin seed oil; No. CAS: 1242417-48-6).

According to another embodiment, the lipophilic form of tyrosine corresponds to a vegetable oil in which the tyrosine has been formulated.

According to a particular embodiment, the vegetable oil is oleic sunflower oil, in particular deodorized. Thus, the OLEOACTIVE TYROSINE BASED Helianthus annus raw material marketed by OLEOS, and corresponding to the INCI designations: Helianthus annuus seed oil (and) tyrosine (and) glyceryl stearate, may be used in the context of the present invention.

In practice, the lipophilic form of tyrosine, such as in oleoyl-tyrosine-based cosmetic ingredients (advantageously at 50% by weight) or tyrosine formulated in vegetable oil, represents between 0.1% and 10% by weight relative to the total weight of the composition, advantageously between 1 and 3%, even more advantageously between 1% and 1.5%.

The composition according to the invention may further comprise active ingredients having depigmenting properties, such as, for example:
lysine azeilate, or other derivatives or salts of azelaic acid;
andrographolide, in particular the extract of Andrographis paniculata corresponding to the INCI designation: Andrographis paniculata leaf extract;
native ascorbic acid (vitamin C) or its derivatives, in particular the derivatives corresponding to the INCI designations: ascorbyl glucoside, ethyl ascorbic acid, ascorbyl methylsilanol pectinate, sodium ascorbyl phosphate and ascorbyl tetraisopalmitate, advantageously ascorbyl glucoside;
arbutin or a plant extract containing it, in particular bearberry extract corresponding to the INCI designation: Arctostaphylos uva-ursi leaf extract;
glabridin or a plant extract containing it, in particular licorice extracts corresponding to the INCI designation: Glycyrrhiza glabra root extract, Glycyrrhiza inflata root extract, Glycyrrhiza uralensis root extract;
biomimetic peptides corresponding to the INCI designations: hexapeptide 2 and/or nonapeptide-1;
an aqueous extract of an algae called Palmaria palmata, in particular the extract corresponding to the INCI designation: Palmaria palmata extract;
4-n-butylresorcinol;
vitamin PP, also called niacinamide or nicotinamide, and its derivatives;
or mixtures thereof.

The composition according to the invention may further comprise active ingredients having healing properties such as, for example, an antimicrobial agent chosen from active ingredients corresponding to the following INCI designations: copper sulfate, zinc sulfate, sodium hyaluronate, Vitis vinifera (grape) vine extract and mixtures thereof.

The composition according to the invention may further comprise active ingredients having sebocorrective, keratolytic, seboregulatory properties and/or anti-acne activity, in order to allow the formulation of solar screening agent products treating acne.

For example, the composition according to the invention may comprise an antimicrobial agent chosen from active ingredients corresponding to the following INCI designations: propyl gallate, dodecyl gallate, Ginkgo biloba leaf extract, bakuchiol, dihydromyricetin, zinc gluconate, salicylic acid and mixtures thereof.

Therefore, the composition according to the invention may further comprise at least one ingredient chosen from the following list:
an agent capable of screening agenting visible light, in particular blue light;
an extract of the algae Laminaria ochroleuca, Blidingia minima or Laminaria saccharina;
an extract of the Zanthoxylum alatum plant;
panthenol;
a cade wood extract;
a Boldo extract;
a meadowsweet extract;
an extract of karanja oil from Pongamia glabra;
linear paraffins;
ATP (adenosine-5 tri-phosphate), Gp4G (diguanosine tetraphosphate) or Ap4A (diadenosine tetraphosphate),
an amino acid chosen from the group consisting of decarboxycarnosine, glutamine and their salts.

The composition according to the invention may further comprise adjuvants such as those usually used in the field of cosmetics, such as preservatives, antioxidants, complexing agents, solvents, perfumes, fillers, bactericides, electrolytes, odor absorbers, coloring materials or even lipid vesicles. The choice of these adjuvants, as well as their concentrations, must be determined in order to prevent them from modifying the properties and advantages sought for the composition of the present invention.

The composition of the invention is for topical application and, more particularly, for application to the skin, lips, hair and/or mucous membranes. Thus, and in the context of the invention, such a composition is intended, in particular, for the protection of the skin and/or integuments, in particular the mucous membranes, lips and hair, against UV radiation.

The composition of the invention may be presented in all the galenic forms normally used in the cosmetic and dermatological fields, such as, for example, but not limited to, in the form of an aqueous solution optionally gelled, a dispersion of the lotion type, a more or less fluid O/W or, conversely, W/O emulsion, or a multiple emulsion such as, for example, a triple emulsion (W/O/W or O/W/O), or even in the form of a vesicular dispersion of ionic (liposomes) and/or non-ionic type, a two-phase composition devoid of emulsifiers and gelling agents whose immiscible phases separate during storage, foam, stick, anhydrous oil, spray or mist.

In a preferred embodiment, the composition according to the invention is a W/O emulsion. In a particular embodiment, the W/O emulsions according to the invention comprise PEG-30 dipolyhydroxystearate (INCI), or polyglyceryl-4 diisostearate/polyhydroxystearate/sebacate as emulsifiers. These raw materials are available from CRODA under the trade name Cithrol DPHS and from EVONIK under the name Isolan GPS, respectively.

In an alternative embodiment, the composition according to the invention is an O/W emulsion. In a particular embodiment, the O/W emulsions according to the invention comprise an emulsifier chosen from the following group of compounds identified by their INCI designation: sodium stearoyl glutamate, potassium cetyl phosphate, glyceryl stearate/PEG-100 stearate and C20-22 alkyl phosphate/C20-C22 alkyl alcohols, tribehenin PEG-20 esters, C14-C22 alcohols/C12-20 alkyl glucoside, cetearyl alcohol/coco-glucoside, polyglyceryl-6 stearate, polyglyceryl-6 behenate and mixtures thereof. These raw materials are available from several suppliers. For example, EMULGIN SG raw materials (INCI: sodium stearoyl glutamate; supplier: BASF); EMULIUM 22 (INCI: tribehenin PEG-20 esters; supplier GATTEFOSSE); SENSANOV WR (INCI: C20-22 alkyl phosphate/C20-C22 alkyl alcohols; supplier: SEPPIC); MONTANOV L (INCI: C14-C22 alcohols/C12-20 alkyl glucoside), AMPHISOL K (INCI: potassium cetyl phosphate; supplier: DSM), MONTANOV 82 (INCI: cetearyl alcohol/coco-glucoside; supplier: SEPPIC); TEGO™ Care PBS 6 MB (INCI: polyglyceryl-6 stearate & polyglyceryl-6 behenate; supplier: EVONIK) may be used in the compositions according to the invention.

According to another aspect, the invention relates to a composition as described above to be used for protection against ultraviolet solar radiation, in particular of wavelength between 100 and 400 nm.

According to a particular embodiment, the invention relates to a composition comprising:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least one organic UVB screening agent or a broad-spectrum organic screening agent absorbing both UVBs and UVAs;
at least one organic UVA screening agent;
   to be used to protect the skin, mucous membranes and/or integuments against UV radiation.

According to a particular embodiment, the composition according to the invention is used to screening agent UV radiation between 280 and 400 nm, in particular between 300 and 380 nm.

According to a particular embodiment, the invention relates to a composition comprising:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two organic screening agents chosen from organic UVB screening agents and/or broad-spectrum organic screening agents absorbing both UVBs and UVAs;
at least one organic UVA screening agent,
   to be used as a UV screening agenting agent, in particular for the wavelength spectrum between 280 and 400 nm, in particular between 300 and 380 nm.

According to a particular embodiment, the invention relates to the use of:
at least one MAA as defined above, in particular chosen from the group consisting of those corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two organic screening agents chosen from organic UVB screening agents and/or broad-spectrum organic screening agents absorbing both UVBs and UVAs; with
at least one organic UVA screening agent;
   to be used for the preparation of a cosmetic composition.

The invention also relates to a cosmetic treatment process consisting of applying to the skin and to the integuments a composition comprising:
at least one MAA as defined above, in particular chosen from the group consisting of the molecules corresponding to the INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as, optionally, the compound corresponding to CAS number 2699128-33-9;
at least two organic screening agents chosen from organic UVB screening agents and/or broad-spectrum organic screening agents absorbing both UVBs and UVAs;
at least one organic UVA screening agent.

The manner in which the invention may be carried out and the resulting advantages will become clearer from the following exemplary embodiments, given by way of example but not limited thereto.

### EXEMPLARY EMBODIMENTS OF THE INVENTION

In the tables below, the percentages indicated are given in mass of product relative to the total mass of the composition.

### Example I—Solar Emulsion

TABLE 1

| | Percentage |
|---|---|
| Ingredients (INCI designation) | (%) |
| Aqua | 52.70 |
| Diethylamino hydroxybenzoyl hexyl benzoate | 10.00 |
| (organic UVA solar screening agent) | |
| Bis-ethylhexyloxyphenol methoxyphenyl triazine | 3.50 |
| (broad-spectrum organic solar screening agent) | |
| Diethylhexyl butamido triazone (broad-spectrum | — |
| organic solar screening agent) | |
| Methoxyphenylimino dimethylhexahydro benzothiazine | 1.00 |
| carboxylic acid (MAA according to the invention) | |
| Hexyl laurate | 10.00 |
| Propanediol | 5.00 |
| C12-15 alkyl benzoate | 4.00 |
| Dicaprylyl carbonate | 3.9988 |
| Polyglyceryl-6 stearate | 2.2875 |
| Glycerin | 2.00 |
| Microcrystalline cellulose | 1.32 |
| 1,2-hexanediol | 1.25 |
| C20-22 alkyl phosphate | 1.10 |
| C20-22 alcohols | 0.90 |
| Caprylyl glycol | 0.25 |
| Polyglyceryl-6 behenate | 0.2125 |
| Sodium citrate | 0.20 |
| Cellulose gum | 0.18 |
| Sodium hydroxide | 0.06 |
| Xanthan gum | 0.04 |
| Tocopherol | 0.0012 |

### Example II—Solar Emulsion

TABLE 2

| | Percentage |
|---|---|
| Ingredients (INCI designation) | (%) |
| Aqua | 51.838 |
| Homosalate (organic UVB solar screening agent) | 9.00 |
| Ethylhexyl methoxycrylene | 6.30 |
| Glycerin | 5.00 |
| Ethylhexyl salicylate (organic UVB solar screening agent) | 4.50 |
| Tribehenin PEG-20 esters | 3.00 |
| Silicon | 3.00 |
| Butyl methoxydibenzoylmethane (organic UVA | 2.70 |
| solar screening agent) | |
| C12-15 alkyl benzoate | 2.50 |
| Dimethicone | 1.72 |
| Butyloctyl salicylate | 1.60 |
| Methoxyphenylimino dimethylcyclohexene glycine | 1.00 |
| (MAA according to the invention) | |
| Benzotriazolyl dodecyl p-cresol | 1.00 |
| Caprylyloxyphenylamino dimethyltetrahydro benzothiazine | 1.00 |
| carboxylic acid (MAA according to the invention) | |
| Vp/eicosene copolymer | 0.80 |
| Cetearyl alcohol | 0.80 |
| C20-22 alkyl phosphate | 0.55 |
| PEG-100 stearate | 0.50 |
| Pentylene glycol | 0.50 |
| Glyceryl stearate | 0.50 |
| C20-22 alcohols | 0.45 |
| Hydroxyethyl acrylate/sodium acryloyldimethyl | 0.396 |
| taurate copolymer | |
| Polysilicon | 0.28 |
| 1,2-hexanediol | 0.25 |
| Caprylyl glycol | 0.25 |
| Xanthan gum | 0.20 |
| Sodium citrate | 0.20 |
| Pentaerythrityl tetra-di-t-butyl hydroxyhydrocinnamate | 0.05 |
| Sodium hydroxide | 0.042 |
| Sorbitan isostearate | 0.027 |
| Polysorbate 60 | 0.027 |
| Aminoethanesulfinic acid | 0.02 |

### Example III—Solar Emulsion

TABLE 3

| | Percentage |
|---|---|
| Ingredients (INCI designation) | (%) |
| Aqua | 57.948 |
| Homosalate (organic UVB solar screening agent) | 9.00 |
| Ethylhexyl methoxycrylene | 6.30 |
| Glycerin | 5.00 |
| Dibutyl adipate | 5.00 |
| Butyl methoxydibenzoylmethane (organic UVA solar | 4.50 |
| screening agent) | |
| Ethylhexyl salicylate (organic UVB solar screening agent) | 4.50 |
| Potassium cetyl phosphate | 3.00 |
| Methoxyphenylimino dimethylhexahydro benzothiazine | 1.00 |
| carboxylic acid (MAA according to the invention) | |
| Sodium stearoyl glutamate | 1.00 |
| Cetearyl alcohol | 0.80 |
| Pentylene glycol | 0.50 |
| Hydroxyethyl acrylate/sodium acryloyldimethyl | 0.396 |
| taurate copolymer | |
| Citric acid | 0.302 |
| Caprylyl glycol | 0.25 |
| 1,2-hexanediol | 0.25 |
| Xanthan gum | 0.20 |
| Sorbitan isostearate | 0.027 |
| Polysorbate 60 | 0.027 |

### Example IV—Solar Emulsion

TABLE 4

| | Percentage |
|---|---|
| Ingredients (INCI designation) | (%) |
| Aqua/water | 48.70 |
| Diethylamino hydroxybenzoyl hexyl benzoate | 10.00 |
| (organic UVA solar screening agent) | |
| Hexyl laurate | 10.00 |
| Propanediol | 5.00 |
| C12-15 alkyl benzoate | 4.00 |
| Diethylhexyl butamido triazone (broad-spectrum | 4.00 |
| organic solar screening agent) | |
| Dicaprylyl carbonate | 3.9988 |
| Bis-ethylhexyloxyphenol methoxyphenyl triazine | 3.500 |
| (broad-spectrum organic solar screening agent) | |
| Polyglyceryl-6 stearate | 2.2875 |
| Glycerin | 2.00 |
| Microcrystalline cellulose | 1.32 |
| 1,2-hexanediol | 1.25 |
| C20-22 alkyl phosphate | 1.10 |
| Methoxyphenylimino dimethylcyclohexenyl ethyl | 1.00 |
| glycinate (MAA according to the invention) | |
| C20-22 alcohols | 0.90 |
| Caprylyl glycol | 0.25 |
| Polyglyceryl-6 behenate | 0.2125 |
| Sodium citrate | 0.20 |
| Cellulose gum | 0.18 |
| Sodium hydroxide | 0.06 |
| Xanthan gum | 0.04 |
| Tocopherol | 0.0012 |

### Example V—Measurement of the Sun Protection Factor (SPF) Obtained With Compositions According to the Invention

### V-1 Objective of the Study

The objective of the study was the development of compositions with a so-called high sun protection factor (SPF), i.e., with an SPF>20, incorporating organic UV screening agents and MAAs according to the invention.

### V-2 Materials and Methods

An in vitro method for measuring the sun protection factor was used. A first UV absorption measurement is carried out on a plate (molded PMMA plate or SB6 sandblasted plate, Helioscreen) covered with 15 μl of glycerin (white), which eliminates absorption by the support.

For Formula 1 and Formula 2, the measurements are carried out using HD6 molded plates, according to the following protocol.

Then, the product studied is taken and deposited in 16 spots on the HD6 molded PMMA plate from Helioscreen (5*5 cm). The weighed quantity is 32.5 mg, which corresponds to a concentration of 1.3 mg/cm². The sample is spread uniformly using a spreading robot (HD-SPREADMASTER-Helioscreen). The plate is then placed in the dark at room temperature for at least 15 min. The plate is introduced into the measuring chamber of the Labsphère UV 2000 S spectrophotometer, to carry out

UV absorption measurements between 290 and 400 nm. It should be noted that a measurement actually corresponds to the average of 9 measurements (9 points on the PMMA plate are measured).

In the cases of Formula 3 to Formula 7, the protocol is identical but SB6 sandblasted plates are used. For these plates, the weighed quantity is 30 mg, which corresponds to a concentration of 1.2 mg/cm².

Theoretical SPFs are calculated for individual screening agents, because in vitro SPF cannot be measured under these conditions. The BASF simulator is used for these measurements (Herzog et al. 2003).

The following formulas are created and tested:

TABLE 5

| | Percentage (%) | |
|---|---|---|
| | | Formula 2 |
| | | (formula |
| | | according to the |
| Ingredients (INCI designation) | Formula 1 | invention) |
| Water | 52.70 | 48.70 |
| Diethylamino hydroxybenzoyl hexyl | 10.00 | 10.00 |
| benzoate (UVA screening agent) | | |
| Bis-ethylhexyloxyphenol methoxyphenyl | 3.50 | 3.50 |
| triazine (broad-spectrum screening | | |
| agent) | | |
| Diethylhexyl butamido triazone | — | 4.00 |
| (broad-spectrum screening agent) | | |
| Methoxyphenylimino | 1.00 | 1.00 |
| dimethylcyclohexene glycine (MAA | | |
| according to the invention) | | |
| Hexyl laurate | 10.00 | 10.00 |
| Propanediol | 5.00 | 5.00 |
| C12-15 alkyl benzoate | 4.00 | 4.00 |
| Dicaprylyl carbonate | 3.9988 | 3.9988 |
| Polyglyceryl-6 stearate | 2.2875 | 2.2875 |
| Glycerin | 2.00 | 2.00 |
| Microcrystalline cellulose | 1.32 | 1.32 |
| 1,2-hexanediol | 1.25 | 1.25 |
| C20-22 alkyl phosphate | 1.10 | 1.10 |
| C20-22 alcohols | 0.90 | 0.90 |
| Caprylyl glycol | 0.25 | 0.25 |
| Polyglyceryl-6 behenate | 0.2125 | 0.2125 |
| Sodium citrate | 0.20 | 0.20 |
| Cellulose gum | 0.18 | 0.18 |
| Sodium hydroxide | 0.06 | 0.06 |
| Xanthan gum | 0.04 | 0.04 |
| Tocopherol | 0.0012 | 0.0012 |

### Table 4: Comparative Formulas Containing Broad-Spectrum and UVA Screening Agents

TABLE 6

| | Percentage (%) | | | | |
|---|---|---|---|---|---|
| | | | Fla 5 | | Fla 7 |
| | | | (formula | | (formula |
| | | | according | | according |
| Ingredients | | | to the | | to the |
| (INCI designation) | Fla 3 | Fla 4 | invention) | Fla 6 | invention) |
| Aqua | 58.948 | 66.948 | 57.948 | 66.948 | 57.948 |
| Homosalate (UVB solar | 9.00 | — | 9.00 | — | 9.00 |
| screening agent) | | | | | |
| Ethylhexyl salicylate | 4.50 | 4.50 | 4.50 | 4.50 | 4.50 |
| (UVB solar screening | | | | | |
| agent) | | | | | |
| Butyl | 4.50 | 4.50 | 4.50 | 4.50 | 4.50 |
| methoxydibenzoylmethane | | | | | |
| (UVA solar screening | | | | | |
| agent) | | | | | |
| Methoxyphenylimino | — | — | — | 1.00 | 1.00 |
| dimethylhexahydro | | | | | |
| benzothiazine carboxylic | | | | | |
| acid (MAA according to | | | | | |
| the invention) | | | | | |
| Methoxyphenylimino | — | 1.00 | 1.00 | 1.00 | 1.00 |
| dimethylcyclohexene | | | | | |
| glycine (MAA according | | | | | |
| to the invention) | | | | | |
| Ethylhexyl | 6.30 | 6.30 | 6.30 | 6.30 | 6.30 |
| methoxycrylene | | | | | |
| Glycerin | 5.00 | 5.00 | 5.00 | 5.00 | 5.00 |
| Dibutyl adipate | 5.00 | 5.00 | 5.00 | 5.00 | 5.00 |
| Potassium cetyl phosphate | 3.00 | 3.00 | 3.00 | 3.00 | 3.00 |
| Sodium stearoyl glutamate | 1.00 | 1.00 | 1.00 | 1.00 | 1.00 |
| Cetearyl alcohol | 0.80 | 0.80 | 0.80 | 0.80 | 0.80 |
| Pentylene glycol | 0.50 | 0.50 | 0.50 | 0.50 | 0.50 |
| Hydroxyethyl | 0.396 | 0.396 | 0.396 | 0.396 | 0.396 |
| acrylate/sodium | | | | | |
| acryloyldimethyl taurate | | | | | |
| copolymer | | | | | |
| Citric acid | 0.302 | 0.302 | 0.302 | 0.302 | 0.302 |
| Caprylyl glycol | 0.25 | 0.25 | 0.25 | 0.25 | 0.25 |
| 1,2-hexanediol | 0.25 | 0.25 | 0.25 | 0.25 | 0.25 |
| Xanthan gum | 0.20 | 0.20 | 0.20 | 0.20 | 0.20 |
| Sorbitan isostearate | 0.027 | 0.027 | 0.027 | 0.027 | 0.027 |
| Polysorbate 60 | 0.027 | 0.027 | 0.027 | 0.027 | 0.027 |

### Table 6: Comparative Formulas Containing UVB and UVA Screening Agents

### IV-3 Results and Discussion

The SPF (sun protection factor) conferred by the different layers is measured:

TABLE 7

| | Number | Average | Standard |
|---|---|---|---|
| Formula | of plates | SPF | deviation |
| Theoretical SPF expected for the | — | 7.5 | — |
| 4.00% diethylhexyl butamido | | | |
| triazone broad-spectrum screening | | | |
| agent | | | |
| Formula 1 | 3 | 5.3 | 0.17 |
| Formula 2 | 3 | 21.01 | 1.01 |

### Table 7: SPF Measurements of Formula 1 and Formula 2

TABLE 8

| | Number | Average | Standard |
|---|---|---|---|
| Formula | of plates | SPF | deviation |
| Theoretical SPF expected for the 9% | — | 4.6 | — |
| homosalate UVB screening agent | | | |
| Formula 3 | 3 | 18.62 | 0.64 |
| Formula 4 | 3 | 12.5 | 0.54 |
| Formula 5 | 3 | 24.11 | 1.49 |
| Formula 6 | 3 | 11.73 | 1.57 |
| Formula 7 | 3 | 25.93 | 1.40 |

### Table 8: SPF Measurements From Formula 3 to Formula 7

In the case of tests relating to broad-spectrum screening agents+UVA screening agent, a very low SPF was obtained for Formula 1 (Fla 1) containing an MAA according to the invention (INCI designation: methoxyphenylimino dimethylcyclohexene glycine), a UVA screening agent and a broad-spectrum screening agent.

The addition of a second broad-spectrum screening agent made it possible to go from an SPF value of 5 to an SPF value of 21, while the theoretical expected contribution for the broad-spectrum screening agent was 7.5.

For tests relating to UVB +UVA screening agents, the addition of a UVB screening agent in Formula 4, containing an MAA according to the invention (INCI designation: methoxyphenylimino dimethylcyclohexene glycine), makes it possible to go from an SPF value of 12.5 at an SPF value of 24.11. The addition of the same screening agent in the same concentration in Formula 6, containing another MAA according to the invention (INCI designation: methoxyphenylimino dimethylhexahydro benzothiazine carboxylic acid) makes it possible to go from an SPF value of 11.73 to an SPF value of 25.93.

The expected theoretical contribution for the 9% homosalate UVB screening agent was 4.6.

The comparison between tests 3 and 5, which differ only by the presence of an MAA according to the invention, validates the sun protection amplifier function of the MAA (SPF 18.62->24.11).

In conclusion, with an equal protection index, the combinations of screening agents and MAA according to the invention make it possible to obtain products characterized by a good protection index (SPF>20), while limiting the introduction of organic screening agents into the formulas thanks to the sun protection amplifier action of MAA.

Thus, the present invention makes it possible to reduce the quantity and concentration of organic screening agents necessary in a sunscreen product, while maintaining a high sun protection factor, i.e., greater than 20. The use of MAAs as anti-UV agents, in combination with organic screening agents, is an eco-compatible solution, eco-responsible for the environment and safe for humans.

### BIBLIOGRAPHIC REFERENCES

Axelstad M., Boberg J., Hougaard K.S., Christiansen S., Jacobsen P.R., Mandrup K.R., Nellemann C., Lund S.P., Hass U. Effects of pre-and postnatal exposure to the UV-screening agent octyl methoxycinnamate (OMC) on the reproductive, auditory and neurological development of rat offspring. Toxicol. Appl. Pharmacol. 250: 278-90 (2011).
Danovaro R., Bongiorni L., Corinaldesi C., Giovannelli D., Damiani E., Astolfi P., Greci L. Pusceddu A. (2008) Sunscreens cause coral bleaching by promoting viral infections. Environ Health Perspect 116(4):441-447.
Herzog, B., Mendrok, C. & Mongiat, S., Müller, S, Osterwalder, U. (2003). The Sunscreen Simulator: A Formulator's Tool to Predict SPF and UVA Parameters. SOFW Journal. 129: 25.
Kinnberg K.L., Petersen G.I., Albrektsen M., Minghlani M., Awad S.M., Holbech B.F., Green J.W., Bjerregaard P., Holbech H. Endocrine-disrupting effect of the ultraviolet screening agent benzophenone-3 in zebrafish, Danio rerio. Environ. Toxicol. Chem. 34: 2833-40 (2015).
Ozáez, I., Morcillo, G., Martínez-Guitarte, J.L. Ultraviolet screening agents differentially impact the expression of key endocrine and stress genes in embryos and larvae of Chironomus riparius. Sci. Total Environ. 557-558: 240-247 (2016).
Petersen, G., Rasmussen, D., Gustavson, K. Study on enhancing the Endocrine Disrupter priority list with a focus on low production volume chemicals Study on enhancing the Endocrine. Revised report to European Commission/DG Environment (2007).
Rehfeld A., et al. Organic Ultraviolet Screening agents Mimic the Action of Progesterone on Human Sperm and Interfere with Sperm Functions. FRI 105-133-Endocrine Disrupting Chemicals and Gene Regulation and Development (posters) (2016).
Schlumpf M., Cotton B., Conscience M., Haller V., Steinmann B., Lichtensteiger W. In vitro and in vivo estrogenicity of UV screens. Environ. Health Perspect. 109: 239-44 (2001).
Schlumpf M., Durrer S., Faass O., Ehnes C., Fuetsch M., Gaille C., Henseler M., Hofkamp L., Maerkel K., Reolon S., Timms B., Tresguerres J.A., Lichtensteiger W. Developmental toxicity of UV screening agents and environmental exposure: a review. Int J Androl. 31: 144-51 (2008).
Szwarcfarb B., Carbone S., Reynoso R., Bollero G., Ponzo O., Moguilevsky J., Scacchi P. Octyl-Methoxycinnamate (OMC), an Ultraviolet (UV) Screening agent, Alters LHRH and Amino Acid Neurotransmitters Release from Hypothalamus of Immature Rats. Exp. Clin. Endocrinol. Diabetes 116: 94-98 (2008).
Zucchi, S., Blüthgen, N., Ieronimo, A., Fent, K. The UV-Absorber benzophenone-4 alters transcripts of genes involved in hormonal pathways in zebrafish (Danio rerio) eleuthero-embryos and adult males. Toxicol. Appl. Pharmacol. 250: 137-46 (2011).

1. A composition comprising:
at least one amino acid analog of mycosporin called “MAA” chosen from the group consisting of molecules A of formula IA or one of its acceptable salts and/or the group consisting of molecules B of formula IB or one of its acceptable salts, said formula IA being: 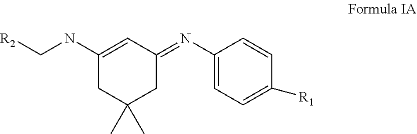
and said formula IB being: 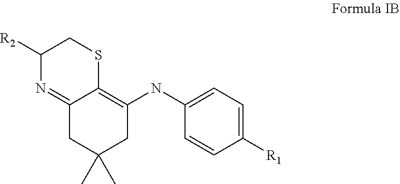
for each of the IA and IB formulas,
R1 is hydrogen; alkyl; alkenyl; alkynyl; aryl; heterocycle; cycloalkyl; alkoxy; alkanoyl; hydroxyl; a sulfo group; a halogen group; a phosphono group; an ester group; a carboxylic acid group; a phenyl group; an amino group; an alkylated fatty acid chain or a polyether;
R2 is alkyl; alkenyl; alkynyl; aryl; heterocycle; cycloalkyl; alkoxy; alkanoyl; a sulfo group; a phosphono group; an ester group; a carboxylic acid group; hydroxyl; or a phenyl group
wherein it further comprises:
at least two organic screening agents chosen from organic UVB screening agents and/or broad-spectrum organic screening agents absorbing both UVBs and UVAs;
at least one organic UVA screening agent.

2. The composition according to claim 1, wherein R1 is an alkoxy group of the [CH₃]—[CH₂]ₙ—O— type, wherein “n” is between 0 and 10.

3. The composition according to claim 1, wherein R2 is a carboxylic acid group of the [COOH]—[CH₂]ₙ— type, wherein “n” is between 0 and 5.

4. The composition according to claim 1, wherein said at least one MAA is chosen from the group consisting of the following INCI designations: methoxyphenylimino dimethylcyclohexene glycine, caprylyloxyphenylamino dimethyltetrahydro benzothiazine carboxylic acid, methoxyphenylimino dimethylhexahydro benzothiazine carboxylic acid, methoxyphenylimino dimethylcyclohexenyl ethyl glycinate, as well as the compound corresponding to CAS number 2699128-33-9.

5. The composition according to claim 1, wherein said at least one organic UVA screening agent is chosen from the group comprising the compounds corresponding to the following INCI designations: butyl methoxydibenzoylmethane, diethylamino hydroxybenzoyl hexyl benzoate, bis-(diethylaminohydroxybenzoyl benzoyl) piperazine, disodium phenyl dibenzimidazole tetrasulfonate, in particular butyl methoxydibenzoylmethane and diethylamino hydroxybenzoyl hexyl benzoate.

6. The composition according to claim 1, wherein said at least one organic UVB screening agent is chosen from the group consisting of the compounds corresponding to the following INCI designations: ethylhexyl salicylate, homosalate, phenylbenzimidazole sulfonic acid and ethylhexyl triazone.

7. The composition according to claim 1, wherein said at least one broad-spectrum organic screening agent is chosen from the group consisting of bis-ethylhexyloxyphenol methoxyphenyl triazine, diethylhexyl butamido triazone, tris-biphenyl triazine, phenylene bis-diphenyltriazine, methylene bis-benzotriazolyl tetramethylbutylphenol and drometrizole trisiloxane.

8. The composition according to claim 1, wherein said at least one MAA represents between 0.1% and 20% by mass relative to the total mass of the composition.

9. The composition according to claim 1, wherein said at least one organic UVB screening agent represents between 1% and 20% by mass relative to the total mass of the composition.

10. The composition according to claim 1, wherein said at least one organic UVA screening agent represents between 1% and 10% by mass relative to the total mass of the composition.

11. The composition according to claim 1, wherein said at least one broad-spectrum organic screening agent absorbing both UVBs and UVAs represents between 1% and 15% by mass relative to the total mass of the composition.

12. The composition according to claim 1, it comprising:
at least two organic UVB screening agents; and
at least one organic UVA screening agent.

13. The composition according to claim 1, it comprising:
at least two broad-spectrum organic screening agents absorbing both UVBs and UVAs; and
at least one organic UVA screening agent.

14. The composition according to claim 1, it comprising:
at least UVB screening agents corresponding to the following INCI designations: octyl salicylate and homosalate;
at least the organic UVA screening agent corresponding to the following INCI designation: butyl methoxydibenzoylmethane.

15. The composition according to claim 1, wherein it comprises:
at least broad-spectrum screening agents absorbing both UVBs and UVAs corresponding to the following INCI designations: diethylhexyl butamido triazone and bis-ethylhexyloxyphenol methoxyphenyl triazine;
-at least one UVA solar screening agent chosen from the group consisting of the compounds corresponding to the following INCI designations: butyl methoxydibenzoylmethane, diethylamino hydroxybenzoyl hexyl benzoate, bis-(diethylaminohydroxybenzoyl benzoyl) piperazine, disodium phenyl dibenzimidazole tetrasulfonate, in particular butyl methoxydibenzoylmethane and diethylamino hydroxybenzoyl hexyl benzoate, advantageously butyl methoxydibenzoylmethane and diethylamino hydroxybenzoyl hexyl benzoate.

16. The composition according to claim 1, wherein it is free from solar screening agents corresponding to the following INCI designations: 4-methylbenzylidene camphor, 3-methylbenzylidene camphor, benzophenone-2, benzophenone-3, benzophenone-4, ethylhexyl methoxycinnamate, isoamyl methoxycinnamate, octocrylene, octyl dimethyl PABA.

17. The composition according to claim 1, wherein it has a sun protection factor called “SPF” equal to or greater than 20.

18. A method for protection against solar radiation, said method comprising applying to skin the composition according to claim 1.

19. The composition according to claim 2, wherein R1 is an alkoxy group of the [CH₃]—[CH₂]ₙ—O— type, wherein “n”=0 or “n”=8.

20. The composition according to claim 3, wherein R2 is a carboxylic acid group of the [COOH]—[CH₂]ₙ— type, wherein “n”=1 or “n”=0.
